(19) United States
(12) Patent Application Publication
(10) Pub. No.: US 2018/0339025 A1
(43) Pub. Date: Nov. 29, 2018

(54) ENZYME COMPOSITION FOR MANAGEMENT OF METABOLIC HEALTH

(71) Applicants: Muhammed Majeed, Edison, NJ (US); Kalyanam Nagabhushanam, East Windsor, NJ (US); Shaheen Majeed, Springville, UT (US)

(72) Inventors: Muhammed Majeed, Edison, NJ (US); Kalyanam Nagabhushanam, East Windsor, NJ (US); Shaheen Majeed, Springville, UT (US)

(21) Appl. No.: 15/986,854

(22) Filed: May 23, 2018

Related U.S. Application Data
(60) Provisional application No. 62/510,858, filed on May 25, 2017.

(51) Int. Cl. A61K 38/47; A61K 38/46; A61K 38/48; A61P 3/00; A61P 3/06; A61K 45/06; A61P 1/14

(52) U.S. Cl. CPC A61K 38/47; C12Y 302/01001; C12Y 302/01108; A61K 38/465; C12Y 302/01004; A61P 1/14; A61K 38/4886; A61P 3/00; A61P 3/06; A61K 45/06; C12Y 304/24028

(57) ABSTRACT

The present invention discloses compositions containing digestive enzymes for the management and maintenance of metabolic health. Specifically, the invention discloses the use of digestive enzymes comprising α-amylase, lactase, lipase, cellulase and Neutral protease or Acid protease in weight management, reducing cholesterol levels, maintaining healthy gut and improving quality of life.

### CROSS-REFERENCE TO RELATED APPLICATION

This is a non-provisional application of U.S. provisional application No. 62/510,858 filed on 25 May 2017.

### BACKGROUND OF THE INVENTION

### Field of the invention

The present invention in general relates to digestive enzymes. More specifically the present invention relates to the use of enzyme composition comprising of α-amylase, lactase, lipase, cellulose and protease for the management and improvement of metabolic health.

### Description of Prior Art

Metabolic syndrome is a cluster or combination of metabolic disorders that increase a person's risk for heart disease, diabetes, and stroke. According to National institutes of Health (NIH), it is a serious global health challenge with 23% of adult population exposed to increased risk of cardiovascular disease, diabetes, and stroke. A person will be at increased risk for cardiovascular problems if he/she presents with these conditions together than any one factor presenting alone (Metabolic Syndrome, National Heart, Lung and Blood institute, U.S. Dept of Health and Human Services, https://www.nhlbi.nih.gov/health-topics/metabolic-syndrome, accessed 10 Mar., 2018). Of the risk factors involved in the development of metabolic syndrome, obesity is considered to be the main cause for the development of chronic conditions ranging from cardiovascular diseases, hypertension, type H diabetes, metabolic syndrome, and stroke to osteoarthritis and cancer.

The microbes present in the gastro-intestinal tract, responsible for maintenance of healthy gut also has a role in the maintaining a good metabolic health (Jansen & Kersten (2015), The role of the gut microbiota in metabolic health, The FASEB Journal Vol. 29, No. 8, pp. 3111-3123). Many clinical studies indicate reduction in the number of probiotic bacteria like Bifidobacterium and Bacteroides and increase in numbers of Staphylococcus, Enterobacteriaceae and Escherichia coli in obese individuals. E. coli numbers were higher in population with excessive weight gain than in population with normal weight (Santacruz et al., Gut microbiota composition is associated with body weight, weight gain and biochemical parameters in pregnant women. Br J Nutr. 2010; 104(1): 83-92). Altering gut microbiota by increasing the probiotic bacteria and reducing pathogenic microbes would be an effective intervention for improving metabolic health.

Though lifestyle modifications, including diet and exercise interventions are the recommended modalities for metabolic syndrome, pharmacotherapy may be considered if the interventions are ineffective for obese individuals with a BMI≥30 or for those with a BMI≥27 when co-morbidities, such as hypertension or type TI diabetes are diagnosed. The drugs that are typically used in the treatment of metabolic syndrome are disclosed by Marvasti et al., Pharmacological management of metabolic syndrome and its lipid complications; Daru. 2010; 18(3); 146-154, However, drugs that can reliably reduce all of the metabolic risk factors are prone to cause undesirable side effects (Anti-Obesity Drugs: A Review about Their Effects and Safety, Jun Goo Kangl and Cheol-Young Park, Diabetes Metab J. 2012 Feb; 36(1): 13-25; Drug therapy of the metabolic syndrome: minimizing the emerging crisis in polypharmacy, Grundy SM, Nat Rev Drug Discov. 2006 Apr; 5(4): 295-309). Atherapeutic alternative which isnatural, safe, efficacious, and sustainable in managing the metabolic syndrome is warranted.

Recent studies have suggested that levels of enzymes like salivary amylase are associated, with metabolic syndrome, obesity, diabetes, and cardiometabolic conditions (Salivary Amylase: Digestion and Metabolic Syndrome, Peyrot des Gachons C. Breslin P A, Curr Diab Rep. 2016 Oct; 16(10): 102), as well as animal model suggesting beneficial effects of digestive enzyme supplementation on scrum cholesterol concentrations (Experimental support for the effects of a probiotic/digestive enzyme supplement on serum cholesterol concentrations and the intestinal microbiome, Ichim T E. Patel A N, Shafer K A, J Transl Med. 2016 Jun 22; 14(1): 184). However, there exist an unmet industrial need for a blend of digestive enzymes complex which improves metabolic function by effectively aiding in reducing/maintaining body weight, BMI, lipid/sugar levels and also aiding in maintaining a healthy gut health thereby improving the quality of life. The present invention solves the above problem by disclosing a blend of digestives enzymes for the management of metabolic syndrome.

The principle objective of the invention is to disclose a blend of digestive enzymes comprising of α-amylase, protease, lipase, cellulase, and lactase for the management and improvement of metabolic health.

It is another objective of the invention to disclose a blend of digestive enzymes comprising of α-amylase, protease, lipase, cellulase, and lactase for maintaining a healthy gut.

It is another objective of the invention to disclose the hypolipidemic effects of a blend of digestive enzymes comprising of α-amylase, protease, lipase, cellulase, and lactase.

The present invention fulfils the aforesaid objective and also provides other related advantages.

### SUMMARY OF THE INVENTION

The present invention pertains to enzyme composition for the management and maintenance of metabolic health. Specifically, the invention discloses the use of enzyme composition comprising α-amylase, lactase, lipase, cellulase and Neutral protease or Acid protease in weight management, reducing cholesterol levels, maintaining a healthy gut and thereby improving quality of Life.

Other features and advantages of the present invention will become apparent from the following more detailed description, taken in conjunction with the accompanying images, which illustrate, by way of example, the principle of the invention.

### DESCRIPTION OF THE MOST PREFERRED EMBODIMENTS

In most preferred embodiment, the invention discloses a method for management and improvement of metabolic health in mammals, said method comprising step of administering effective concentration of enzyme blend comprising of α-amylase, cellulase, lipase, lactase and neutral protease or acid protease to mammals in need of such therapy. In a related embodiment, the improvement in metabolic health is brought about by a) reducing and/or maintaining constant body weight and BMI, b) reducing lipid levels, and c) improving gut health and quality of life. In another related embodiment, the effective concentration of the enzyme blend, is 20-200 mg, with concentrations of the individual enzymes being a) α-amylase: not less than 24000 DU/g, b) cellulase: not less than 1100 CU/g, c) lipase: not less than 200 FIP/g, d) lactase: not less than 4000 ALU/g and e) neutral or acid protease: not less than 6000 PC/g. In yet another related embodiment, the composition is formulated with pharmaceutically/nutraceutically acceptable excipients, adjuvants, diluents or carriers and administered orally as a stand-alone or in combination with standard treatment, in the form of tablets, capsules, syrups, gummies, powders, suspensions, emulsions, chewables, candies and eatables. In another related embodiment, the standard treatment involves administration of one or more drugs selected from the group consisting of antibiotics, statins, cholesterol absorption inhibitors, Insulin sensitizing agents, GLP-1 receptor agonists and DPP-4 inhibitors. In another related embodiment, the mammal is preferably human.

In another preferred embodiment, the invention discloses a method for the reduction of hyperlipidemia in mammals, said method comprising step of administering effective concentration of enzyme blend comprising of α-amylase, cellulase, lipase, lactase and neutral protease or acid protease to said mammals to bring about the effect of reduction in the levels of a) total cholesterol, b) LDL and LDL-Cholesterol, and c) cholesterol:HDL ratio in said mammals. In another related embodiment, the effective concentration of the enzyme blend is 20-200 mg, with the concentration of the individual enzymes being a) α-amylase: not less than 24000 DU/g, b) cellulase: not less than 1100 CU/g, c) lipase; not less than 200 FIP/g, d) lactase; not less than 4000 ALU/g and e) neutral or acid protease: not less than 6000 PC/g In yet another related embodiment, the composition is formulated with pharmaceutically/nutraceutically acceptable excipients, adjuvants, diluents or carriers and administered orally as a stand-alone or in combination with standard treatment, in the form of tablets, capsules, syrups, gummies, powders, suspensions, emulsions, chewables, candies and eatables. In another related embodiment, the standard treatment involves administration of one or more drugs selected from the group consisting of antibiotics, statins, cholesterol absorption inhibitors, Insulin sensitizing agents, GLP-1 receptor agonists and DPP-4 inhibitors, In another related embodiment, the mammal is preferably human.

In a yet another preferred embodiment, the invention discloses a method for the improvement in gut health in mammals, said method comprising steps of administering effective amounts of enzyme blend comprising of α-amylase, cellulase, lipase, lactase and neutral protease or acid protease to mammals showing symptoms of gastrointestinal discomfort. In a related embodiment, the symptoms associated with gastrointestinal discomfort include presence of E. coli in stool, gastric reflux, abdominal pain, indigestion, diarrhoea and constipation. In another related embodiment, the effective concentration of the enzyme blend is 20-200 mg, with concentrations of the individual enzymes being a) α-amylase: not less than 24000 DU/g, b) cellulase: not less than 1100 CU/g, c) lipase: not less than 200 FIP/g, d) lactase: not less than 4000 ALU/a and e) neutral or acid protease: not less than 6000 PC/g. In yet another related embodiment, the composition is formulated with pharmaceutically/nutraceutically acceptable excipients, adjuvants, diluents or carriers and administered orally as a stand-alone or in combination with standard treatment, in the form of tablets, capsules, syrups, gummies, powders, suspensions, emulsions, chewables, candies and eatables. In another related embodiment. the standard treatment involves administration of one, or more drugs selected from the group consisting of antibiotics, statins, cholesterol absorption inhibitors, Insulin sensitizing agents, CLP-1 receptor agonists and DPP-4 inhibitors. In another related embodiment, the mammal is preferably human.

The aforesaid most preferred embodiments incorporating the technical features and technical effects of instant invention, are explained through illustrative examples herein under.

### EXAMPLE 1

### Methods

Study Objectives:

The objective of the study is to assess the efficacy of enzyme blend in the management of metabolic health. The primary objectives include evaluating the change in the body weight and the body mass index (BMI) by the enzyme blend and changes in biochemical parameters (Serum lipids, Stool Analysis). The changes in the gut health will be evaluated using a questionnaire. The study also assesses the quality of life as a secondary objective.

Selection of Subjects

Number of subjects: 30

### Nature of Population

Male and female healthy subjects between the age group of 18-65 years with BMI between 30 to 34.9 kg/m² were screened for this study. The screening procedures included medical history, medication history, treatment history, co-morbid conditions, physical examination, vital signs and urine pregnancy test for women, lipid profile, hematology, biochemistry and stool analysis. Subjects who met the inclusion criteria or for whom the abnormalities in laboratory parameters are not clinically significant were selected for the study.

Inclusion Criteria

Subject who met the below mentioned inclusion criteria were enrolled in the study

Male and/or female.
Age between 18 to 65 years.
BMI between 30 to 34.9 Kg/m²
Willing to come for regular followup visits.
Able to give written informed consent.

Exclusion Criteria

Subject who met any of the below mentioned exclusion criteria were excluded from the study.

Intake of over the counter weight loss agents, centrally acting appetite suppressants in the previous six months.
Pathophysiologic/genetic syndromes associated with obesity (Cushing's syndrome, Turner's syndrome, Praderwilli syndrome).
Patients on prolonged (>6weeks) medication with corticosteroids, antidepressants, anticholinergics, etc. or any other drugs that may have an influence on the outcome of the study.
Patients suffering from major systemic illness necessitating long term drug treatment (Rheumatoid arthritis, Tuberculosis, Psycho-Neuro-Endocrinal disorders, etc.)
Symptomatic patient with clinical evidence of Heart failure.
History of HIV and other viral infections.
Alcoholics and/or drug abusers.
Prior surgical therapy for obesity
H/o hypersensitivity to any of the trial drugs or their ingredients.
Pregnant/lactating, woman.

Study Design

Subjects were screened only after obtaining their informed consent. Participants who met the inclusion criteria and did not violate the exclusion criteria were administered with a capsule containing the enzyme blend(DigeZyme®—Sabinsa Corporation, USA) for a period of 90 days. Table 1 provides the constituents on the enzyme blend

TABLE 1
Active ingredient of Capsule

| S. No. | Enzyme | Source |
|---|---|---|
| 1. | Alpha-amylase | Aspergillus oryzae |
| 2. | Lactase | Aspergillus oryzae |
| 3. | Lipase | Rhizopus oryzae |
| 4. | Cellulase | Trichoderma longibrachiatum, |
| | | Trichoderma reesei |
| 5. | Neutral Protease | Bacillus subtilis |
| | Acid Protease | Aspergillus oryzae |

Screening Visit

A total of 30 participants were enrolled after obtaining informed consent, medical & medication history, treatment history, co-morbid conditions, physical examination, demographic data, vital signs, blood sample for laboratory analysis like haematology, lipid profile & biochemistry, urine sample for urine pregnancy test & stool analysis (routine examination , culture count & sensitivity for E. Coli & Bifidogenic bacteria) & Anthropometric measurements (the size, weight, and proportions of the body hips & waist) were performed and recorded.

Day 0 (Visit 1)

Evaluations for Inclusion & exclusion criteria were carried out and eligible subjects were enrolled into the study. Subjects were instructed on their daily dose of study supplement. Subjects were provided with study visit plan. Vital signs & anthropometric measurements were documented in the respective CRFs. Concomitant medications, if any, were recorded.

Study supplements were provided to all subjects as per the randomization code, subjects were instructed to take daily the enzyme blend capsules on days not coming to the clinic. Assessments using subjective questionnaires were performed.

Day 45 (Visit 2)

Study supplements were provided to all subjects as per the randomization code, subjects were instructed to take daily the enzyme blend supplement capsules on days not corning to the clinic. Demographics, Physical examination, vital signs & anthropometric measurements & lipid profile were documented in the respective CRFs. Photographs of the subjects were taken. Adverse Events and concomitant medications, if any, were recorded. Assessments using subjective questionnaires were performed.

Day 90 (Final Visit)

Demographics, Physical examination, vital signs & anthropometric measurements were documented in the respective CRFs. Urine pregnancy test for women were performed. Stool analysis, haematology; serum biochemistry and lipid profile tests were performed. Adverse Events and concomitant medications, if any, were recorded by the site personnel. Concomitant medication, if any, was recorded. Photographs of the subjects were taken. Assessments using subjective questionnaires were performed.

Follow Up Visit

Telephonic Follow up (15 days from the final visit for the overall general well being of the subject)

Efficacy and Safety Variables

### Efficacy Evaluation

Efficacy was analysed by

Changes in the body weight and the body mass index.

Changes in the lab investigations (Serum lipids, Stool Analysis).

Changes in the gut health questionnaire

Improvement in Quality of Life as per Quality of Life Questionnaire

All the subjects enrolled in the study were evaluated for the effect of study supplement in healthy volunteers for metabolic health during the end of study or at the time of discontinuation of the treatment in comparison with baseline.

Safety Data

Safety measurements included monitoring of AEs (including worsening of gastrointestinal symptoms), physical examination results and vital signs were performed

Statistical Methods

The primary efficacy measure was the decrease in the body weight and BMI, changes in the lab investigations (serum lipids & stool analysis), changes in the gut health questionnaire from baseline to end of the study. Secondary efficacy measures included Improvement in Quality of Life as per Quality of Life Questionnaire, tolerability of the study drug by assessing for the adverse events and other physical signs or symptoms by the Investigator. For statistical analysis purposes the baseline values was compared to that of all visit values by statistical analysis in terms of mean, standard deviation. The results between the first and the subsequent visiting were analyses using paired t test.

### EXAMPLE 2

### Results

Baseline and Demographic Characteristic

Thirty healthy subjects with BMI between 30 to 34.9 Kg/m² who were deemed eligible to have met the study requirements were administered the digestive enzyme blend for a period of 90 days. The demographic data and baseline characteristics of the enrolled patient population are summarized in Table 2.

TABLE 2
Baseline and demographic characteristic

| | | Enzyme |
|---|---|---|
| Parameter | Statistics | blend (N = 30) |
| Age (years) | Mean ± SD | 36 ± 9.77 |
| | Median | 35 |
| | Min; Max | 22, 59 |
| Gender, n (%) | Male | 16 (53.33) |
| | Female | 14 (46.67) |
| Body weight (kg) | Mean ± SD | 80 ± 9.62 |
| | Median | 82 |
| | Min; Max | 64, 98 |
| Height (cm) | Mean ± SD | 159 ± 9.95 |
| | Median | 162 |
| | Min; Max | 143, 175 |

Efficacy Evaluation

Decrease in the Body Weight and the Body Mass Index (BMI)

The changes in the body weight and the body mass index (BMI) are presented in Table 3.

TABLE 3
Changes in bodyweight and BMI

| | Body weight (kg) | BMI (kg/m²) |
|---|---|---|
| Baseline (Day 0) | 80.12 ± 9.62 | 31.77 ± 1.07 |
| Visit 2 (Day 45) | 79.55 ± 9.59* | 31.55 ± 1.11* |
| Visit 3 (Day 90) | 78.67 ± 9.67* | 31.17 ± 1.73* |

*Statistically significant (P < 0.05) compared to baseline (Paired t-test)

The results indicated that the digestive enzyme supplement showed a slight decrease in bodyweight and BMI. The enzyme blend also helps in maintaining constant body weight with no signs of increase in BMI.

Changes in the Lab Investigations

The lipid profile was estimated at the baseline and during each visit. The results are tabulated in table 4.

TABLE 4
Lipid profile in subjects supplemented with enzyme blend

| | Baseline | | Visit 2 | | Visit 3 | |
|---|---|---|---|---|---|---|
| | (Day 0) | | (Day 45) | | (Day 90) | |
| Total cholesterol | 188.97 ± | 32.11 | 182.67 ± | 28.45* | 176.03 ± | 26.73* |
| (mg/dl) | | | | | | |
| Triglycerides | 157.47 ± | 57.59 | 169.27 ± | 57.43 | 157.53 ± | 48.88 |
| (mg/dl) | | | | | | |
| HDL (mg/dl) | 38.5 ± | 8.45 | 41.57 ± | 9.58 | 37.23 ± | 7.33 |
| LDL (mg/dl) | 129.27 ± | 26.32 | 120.47 ± | 25.95* | 113.53 ± | 25.05* |
| VLDL (mg/dl) | 21.2 ± | 10.49 | 21.57 ± | 8.30 | 27.37 ± | 20.45 |
| Cholesterol:HDL | 5.08 ± | 1.20 | 4.57 ± | 1.05* | 4.79 ± | 1.27* |
| ratio | | | | | | |

*Statistically significant (P < 0.05) compared to baseline (Paired t-test)

The results indicated that the enzyme blend reduced the total cholesterol. LDL levels and cholesterol:HDL ratio

Stool Analysis

In many Clinical studies the reduced numbers of Bifidobacterium and Bacteroides and increased numbers of Staphylococcus, Enterobacteriaceae and Escherichia coli were detected in overweight compared with normal-weight population. E. coli numbers are reported to be higher in population with excessive weight gain than in population with normal weight. The present study analysed the E. coli numbers in stool and its relationship with body weight and weight gain. The E. coli numbers in stools was tabulated in Table 5.

TABLE 5
Presence of E. coli in stool

| | E. coli cells (No.) |
|---|---|
| Baseline (Day 0) | 70.83 ± 15.10 |
| Visit 3 (Day 90) | 63.08 ± 14.54* |

*Statistically significant (P < 0.05) compared to baseline (Paired t-test)

E. coli numbers was found to be lower in visit 3 indicating a change in the gut microbiota as a result of enzyme supplementation. This reduction correlated with the change in body weight in BMI indicating that the change in gut microbiota reduces bodyweight and helps in the management of metabolic health.

The change in gut microbiota also resulted in the improvement of gut health, which is indicated below:

Gut Health Assessment

The gut health questionnaire helps in determining the imbalance in the digestive system. In the present study, it was assessed in baseline, visit 3 and final visit and the result are tabulated in table 6.

TABLE 6
Gut health assessment score

| | Gut health assessment Score |
|---|---|
| Baseline (Day 0) | 26.57 ± 11.35 |
| Visit 2 (Day 45) | 20.13 ± 6.45* |
| Visit 3 (Day 90) | 14.33 ± 4.78* |

*Statistically significant (P < 0.05) compared to baseline (Paired t-test)

In the baseline visit the mean of Gut Health Assessment score was found to be 26.57+11.35 and significantly reduced to 20.13±6.45 in visit 2 and 14.33±4.78 in final visit indicating that the enzyme supplement significantly improved the gut health.

Gastrointestinal Symptom Rating Scale (GSRS)

The GSRS is a disease-specific instrument of 15 items combined into five symptom clusters depicting reflux, abdominal pain, indigestion, diarrhea and constipation. The GSRS has a four-point graded scale where 0 represents absence of troublesome symptoms and 3 represent very troublesome symptoms. In the current investigation, the GSRS was in baseline, visit 2 and final visit. The results are indicated in table 7.

TABLE 7
Change in Gastrointestinal Symptom Rating Scale

| | Gut health assessment Score |
|---|---|
| Baseline (Day 0) | 6.9 ± 2.59 |
| Visit 2 (Day 45) | 5.13 ± 1.78* |
| Visit 3 (Day 90) | 3.57 ± 1.48* |

*Statistically significant (P < 0.05) compared to baseline (Paired t-test)

The results indicated a significant reduction in GSRS score in the follow up visits when compared to the initial visit indicating that the enzyme blend supplement improved the gastrointestinal function.

Improvement in Quality of Life

Physically, sonic of the problems associated with obesity are hypertension, coronary arteriosclerosis, elevated cholesterol, type 2 diabetes, joint problems, stroke, and certain types of cancers. Psychologically, obesity is associated with a myriad of problems including lower self-concept, negative self evaluation, and decreased self-image. Socially, obese individuals often encounter discrimination and prejudice, which further perpetuate negative economic and social consequences. In general, obesity is associated with decrements in overall quality of life whether it is physical, psychological, or social. The lower the score, the more quality of life was thought to be impaired. The impact of being overweight and obese was studied on Baseline, Visit 2 and Visit 3 using a questionnaire. The results are presented in Table 8.

TABLE 8
Mean change in Quality of Life

| | Quality of life score |
|---|---|
| Baseline (Day 0) | 30.93 ± 3.42 |
| Visit 2 (Day 45) | 31.4 ± 3.09 |
| Visit 3 (Day 90) | 35.23 ± 2.82* |

*Statistically significant (P < 0.05) compared to baseline (Paired t-test)

The results indicated that the enzyme blend significantly improved the quality of life.

Overall, supplementation of the enzyme blend containing α-amylase, protease, lipase, cellulose, and lactase, was effective in decreasing/maintaining body weight and BMI, improving gut health and gastrointestinal efficiency by reducing E. coli and alleviating the symptoms of gut disorders. The study indicated that the enzyme blend can be effectively used as a supplement for the management and maintenance of metabolic health. Since, dietary supplements also improve the efficacy of the treatment drugs, the enzyme blend can also be supplemented with the standard drugs used for the treatment of metabolic syndrome.

### EXAMPLE 3

### Compositions/Formulations Containing Digestive Enzymes

Tables 9-13 provide illustrative examples of formulations containing digestive enzyme blend. The digestive enzyme blend disclosed in the current application is available in the name DigeZyme®, from Sabina Corporation, USA.

TABLE 9
DigeZyme ® Capsule

Active Ingredients
DigeZyme ® 20-200 mg containing
α-amylase 24,000 DU/g, lactase 4000 LAC/g, cellulase 1100 CU/g,
lipase 200 FIP/g, acid protease 10,000 HUT/g
Excipients
Maltodextrin, Magnesium stearate

TABLE 10
DigeZyme ® Capsule

Active Ingredients
DigeZyme ® 20-200 mg containing
α-amylase 24,000 DU/g, lactase 4000 LAC/g, cellulase 1100 CU/g,
lipase 200 FIP/g, neutral protease 6000 PC/g
Excipients
Maltodextrin, Magnesium stearate

TABLE 11
DigeZyme ® Capsule

Active Ingredients
DigeZyme ® 20-200 mg containing
α-amylase 24,000 DU/g, lactase 4000 LAC/g, cellulase 1100 CU/g,
lipase 200 FIP/g, neutral protease 7000 PC/g
Excipients
Maltodextrin, Magnesium stearate

TABLE 12
DigeZyme ® Capsule

Active Ingredients
DigeZyme ® 20-200 mg containing
α-amylase 24,000 DU/g, lactase 30,000 LAC/g, cellulose 1100 CU/g,
lipase 200 FIP/g, neutral protease 7000 PC/g
Excipients
Maltodextrin, Magnesium stearate

TABLE 12
DigeZyme ® Drink Mix

Active Ingredients
DigeZyme ® (α-amylase, lactase, lipase, cellulase, Neutral protease or
Acid protease) - 20 to 200 mg
Beet root extract, Taurine, Vitamin B6, Vitamin B 12
Excipients
Maltodextrin, Citric acid, Sucralose, Flavouring agents

The above formulations are merely illustrative examples, any formulation containing the above active ingredient intended for the said purpose will be considered equivalent.

Other modifications and variations to the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention.

We claim:

1. A method for management and improvement of metabolic health in mammals, said method comprising step of administering effective concentration of enzyme blend comprising of α-amylase, cellulase, lipase, lactase and neutral protease or acid protease to mammals in need of such therapy.

2. The method as in claim 1, wherein the improvement in metabolic health is brought about by a) reducing and/or maintaining constant body weight and BMI, b) reducing lipid levels, and c) improving gut health and quality of life.

3. The method as in claim 1, wherein the effective concentration of the enzyme blend is 20-200 mg, with concentrations of the individual enzymes being a) α-amylase: not less than 24000 DU/g, b) cellulase: not less than 1100 CU/g, c) lipase: not less than 200 FIP/g, d) lactase: not less than 4000 ALU/g and e) neutral or acid protease: not less than 6000 PC/g.

4. The method as in claim 1, wherein the composition is formulated with pharmaceutically/nutraceutically acceptable excipients, adjuvants, diluents or carriers and administered orally as a stand-alone or in combination with standard treatment, in the form of tablets, capsules, syrups, gummies, powders, suspensions, emulsions, chewables, candies and eatables.

5. The method as in claim 1, wherein the standard treatment involves administration of one or more drugs selected from the group consisting of antibiotics, statins, cholesterol absorption inhibitors, Insulin sensitizing agents, GLP-1 receptor agonists and DPP-4 inhibitors.

6. The method as in claim 1, wherein the mammal is preferably human.

7. A method for reducing hyperlipidemia in mammals, said method comprising step of administering effective concentration of enzyme blend comprising of α-amylase, cellulase, lipase, lactase and neutral protease or acid protease to said mammals to bring about the effect of reduction in the levels of a) total cholesterol, b) LDL and LDL-Cholesterol, and c) cholesterol:HDL ratio.

8. The method as in claim 7, wherein the effective concentration of the enzyme blend is 20-200 mg, with concentrations of the individual enzymes being a) α-amylase: not less than 24000 DU/g, b) cellulase: not less than 1100 CU/g, c) lipase: not less than 200 FIP/g, d) lactase: not less than 4000 ALU/g and e) neutral or acid protease: not less than 6000 PC/g.

9. The method as in claim 7, wherein the composition is formulated with pharmaceutically/nutraceutically acceptable excipients, adjuvants, diluents or carriers and administered orally as a stand-alone or in combination with standard treatment, in the form of tablets, capsules, syrups, gummies, powders, suspensions, emulsions, chewables, candies and eatables.

10. The method as in claim 7, wherein the standard treatment involves administration of one or more drugs selected from the group consisting of antibiotics, statins, cholesterol absorption inhibitors, Insulin sensitizing agents, GLP-1 receptor agonists and DPP-4 inhibitors.

11. The method as in claim 7, wherein the mammal is preferably human.

12. A method for the improvement in gut health in mammals, said method comprising steps of administering effective amounts of enzyme blend comprising of α-amylase, cellulase, lipase, lactase and neutral protease or acid protease to mammals showing symptoms of gastrointestinal discomfort.

13. The method as in claim 12, wherein the symptoms associated with gastrointestinal discomfort include presence of E. coli in stool, gastric reflux, abdominal pain, indigestion, diarrhoea and constipation.

14. The method as in claim 12, wherein the effective concentration of the enzyme blend is 20-200 mg, with the concentration of the individual enzymes being a) α-amylase: not less than 24000 DU/g, b) cellulase: not less than 1100 CU/g, c) lipase: not less than 200 FIP/g, d) lactase: not less than 4000 ALU/g and e) neutral or acid protease: not less than 6000 PC/g.

15. The method as in claim 12, wherein the composition is formulated with pharmaceutically/nutraceutically acceptable excipients, adjuvants, diluents or carriers and administered orally as a stand-alone or in combination with standard treatment, in the form of tablets, capsules, syrups, gummies, powders, suspensions, emulsions, chewables, candies and eatables.

16. The method as in claim 12, wherein the standard treatment involves administration of one or more drugs selected from the group consisting of antibiotics, statins, cholesterol absorption inhibitors, Insulin sensitizing agents, GLP-1 receptor agonists and DPP-4 inhibitors.

17. The method as in claim 12, wherein the mammal is preferably human.
